SEQUENCE LISTING (2 sequences)

SEQ ID NO: 1 - protein, 6 aa, Homo sapiens
GSSFLS

SEQ ID NO: 2 - protein, 6 aa, Artificial Sequence
  linker
LSPEHQ

(19) United States
(12) Patent Application Publication
(10) Pub. No.: US 2022/0298216 A1
(43) Pub. Date: Sep. 22, 2022

(54) GHRELIN O-ACYLTRANSFERASE (GOAT) IMAGING AGENTS

(71) Applicant: James L. Hougland, Fayetteville, NY (US)

(72) Inventor: James L. Hougland, Fayetteville, NY (US)

(73) Assignee: SYRACUSE UNIVERSITY, SYRACUSE, NY (US)

(21) Appl. No.: 17/832,813

(22) Filed: Jun. 6, 2022

Related U.S. Application Data
(63) Continuation of application No. 16/567,714, filed on Sep. 11, 2019.
(60) Provisional application No. 62/730,653, filed on Sep. 13, 2018.

(51) Int. Cl. C07K 14/63; G01N 33/58; G01N 33/573; G01N 33/60

(52) U.S. Cl. CPC C07K 14/63; G01N 33/582; G01N 33/573; G01N 33/60; G01N 2333/63; G01N 2333/91051

(57) ABSTRACT

Imaging agents that can bind to ghrelin O-acyltransferase (GOAT) without binding to the ghrelin receptor (GHS-R1a). The imaging agents comprise a base structure for selective binding to GOAT that is coupled via an amino acid linker to a chemical group to enable imaging such as a fluorescent label, radioactive tracer, or metal chelator. For example, the imaging agent may comprise a ghrelin substrate mimetic inhibitor incorporating an unmodified 2,3-diaminopropanoic acid (Dap) group at the site analogous to serine 3. These agents enable specific detection and imaging of GOAT versus the GHS-R1a receptor in a variety of biological contexts.

### CROSS-REFERENCE TO RELATED APPLICATIONS

The present application claims priority to U.S. Non-Provisional application Ser. No. 16/567,714, filed on Sep. 11, 2019, which claimed priority to U.S. Provisional App. No. 62/730,653, filed on Sep. 13, 2018.

### BACKGROUND OF THE INVENTION

### 1. Field of the Invention

The present invention relates to biological imaging agents and, more specifically, to imaging agents that can bind to and image ghrelin O-acyltransferase (GOAT) without binding to the ghrelin receptor (GHS-R1a).

### 2. Description of the Related Art

Ghrelin is a 28 amino acid peptide hormone which plays a central role in regulating energy balance within the body. Ghrelin signaling exerts this regulation through its involvement in multiple physiological processes including hunger, adipogenesis, glucose metabolism, and insulin secretion and sensitivity. Ghrelin's involvement in appetite control and energy regulation in multiple diseases has led to proposed connections to diseases such as diabetes, obesity, anorexia nervosa, and hyperphagia in patients with Prader-Willi syndrome. Beyond energy homeostasis, ghrelin has been linked to neurological processes including learning and memory and may also impact addictive behaviors.

Like other peptide hormones, ghrelin requires multiple processing steps in the process of maturation prior to secretion. In addition to several proteolytic cleavages to liberate the 28-amino acid ghrelin from the 117-amino acid precursor preproghrelin, ghrelin also undergoes a unique posttranslational modification wherein a seine residue near the N-terminus is esterified with an octanoyl group. This rare modification is essential for ghrelin to bind and activate the GHS-R1a receptor following secretion into circulation. In addition to its cognate receptor, ghrelin interacts with other biomolecules within the bloodstream such as autoantibodies, lipoproteins, and esterases. These interactions play roles in trafficking ghrelin and regulating ghrelin signaling through conversion of ghrelin to desacyl ghrelin by octanoyl ester hydrolysis. Recent reports supporting desacyl ghrelin re-acylation by bone marrow adipocytes and hypothalamic neurons suggest that the ghrelin/desacyl ghrelin signaling system may be more complex and dynamic than originally proposed.

Ghrelin octanoylation is catalyzed by the enzyme ghrelin O-acyl-transferase (GOAT), a member of the membrane-bound O-acyltransferase (MBOAT) enzyme superfamily. While the majority of MBOAT family members modify small molecule substrates, GOAT is one of three MBOAT, along with Porcupine (PORCN) and Hedgehog acyltransferase (Hhat), which acylate protein substrates. There is substantial interest in inhibitor development targeting these enzymes, but the challenges of studying these topologically complex integral membrane enzymes have limited our understanding. For example, there are extremely limited options for specifically detecting the presence of GOAT in biological samples, complicated by potential cross-reactivity with the ghrelin receptor (GHS-R1a). Accordingly, there is a need for imaging agents that can bind to and image GOAT without binding to GHS-R1a.

### BRIEF SUMMARY OF THE INVENTION

The present invention comprises a series of peptide-based molecules, derived from a new class of GOAT inhibitors, which specifically bind to GOAT and thus serve as agents for imaging and detecting GOAT without binding to GHS-R1a. This present invention also allows for simultaneous imaging of GOAT and GHS-R1a.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of an imaging agent for ghrelin O-acyl-transferase (GOAT) according to the present invention.

FIG. 2 is a schematic of the N- and C-terminal length dependence of Dap-containing ghrelin mimetic peptide GOAT inhibitors;

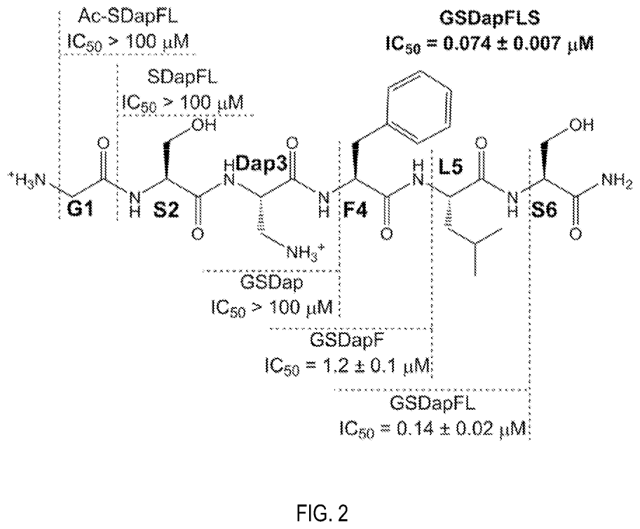

FIG. 3 is a schematic of a generic platform for the design of imaging agents for ghrelin O-acyl-transferase (GOAT) according to the present invention;

FIG. 4 is a chart of certain compounds according to the present invention and the IC₅₀ values for those compounds in GHS-R1A and GOAT assays;

FIG. 5 is a graph of hGOAT activity and inhibition in the presence of SulfoCy5-3, a fluorescently labeled version of the proposed GOAT imaging agent according to FIGS. 3 and 4;

FIG. 6A is a series of images of cell membrane binding in PC3 (GOAT positive) cells; and

FIG. 6B is a series of images of peptide internalization in PC3 (GOAT positive) and HEK293 (GOAT negative) cells using a fluorescently labeled GOAT imaging agent per FIG. 4.

### DETAILED DESCRIPTION OF THE INVENTION

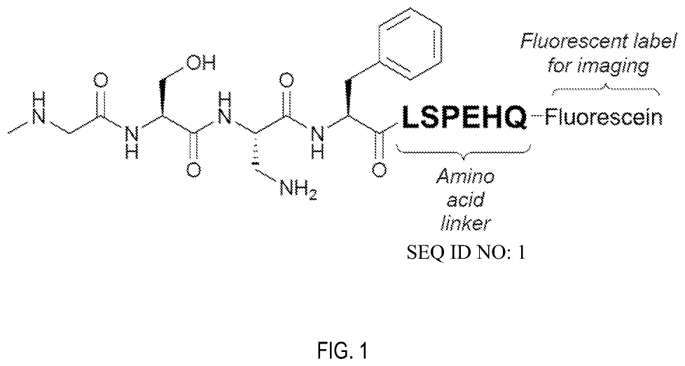

Referring to the figures, wherein like numeral refer to like parts throughout, there is seen in FIG. 1 a schematic of a GOAT imaging agent according to the present invention that is a ghrelin substrate mimetic inhibitor based on the first six amino acids of ghrelin, GSSFLS (SEQ ID NO: 1), that has a sarcosine substitution at the G1 position and an unmodified 2,3-diaminopropanoic acid (Dap) group at the site analogous to serine 3. As seen in FIG. 1, the imaging agent is coupled via an amino acid linker LSPEHQ (SEQ ID NO: 2) to a fluorescent label for imaging, thereby resulting in the complex SarSDapFLSPEHQ-Fluorescein.

The present invention stems from comparison between the structure-activity relationships governing ghrelin binding to GOAT and to the GHS-R1a receptor. Proceeding from the N-terminus of ghrelin, binding to both GOAT and GHS-R1a is severely diminished by acetylation of the N-terminal amino group of ghrelin. A sarcosine substitution at the G1 position leads to a >25-fold loss in binding affinity for the ghrelin receptor as reflected by IC₅₀ values in a competition binding assay, while the same substitution strengthens binding to GOAT by 60 percent, as seen below in Table 1:

TABLE 1
Impact of nitrogen methylation on Dap peptide
inhibitor potency against hGOAT.

| Methylation site(s) | Peptide sequence | IC₅₀ (μM) |
|---|---|---|
| none | GSDapFL | 0.14 ± 0.02 |
| G1 | SarSDapFL | 0.088 ± 0.001 |
| S2 | G_{N·Me}SDapFL | >100 |
| F4 | GSDap_{N·Me}FL | 0.097 ± 0.013 |
| L5 | GSDapF_{N·Me}L | 0.062 ± 0.009 |
| G1, F4 | SarSDap_{N·Me}F | 1.5 ± 0.1 |
| G1, F4, L5 | SarSDap_{N·Me}F_{N·Me}L | 6 ± 1 |

The marked differences in ligand binding requirements between GOAT and GHS-R1a, particularly at the G1 and S3 positions of ghrelin-derived peptides, support the potential for designing molecules that specifically target either of these ghrelin-interacting proteins for use in studying and modulating the ghrelin signaling pathway.

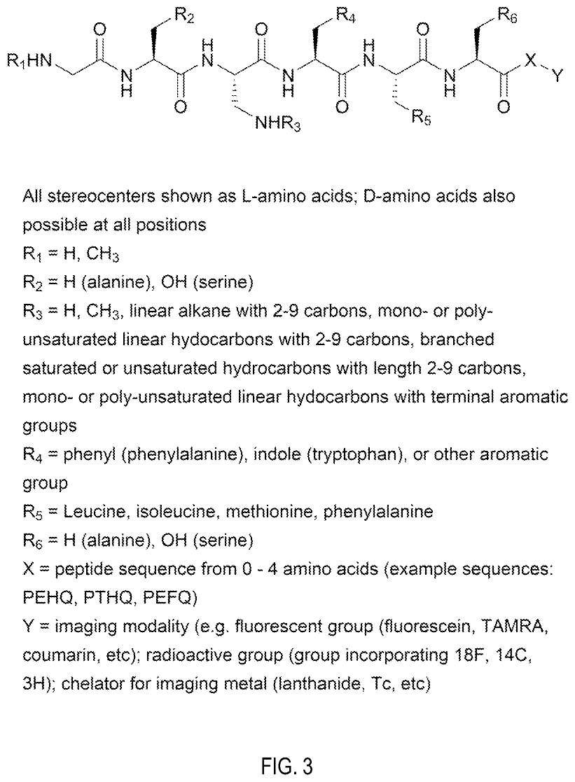

There is seen in FIG. 3 a schematic of alternative approaches for designing a GOAT imaging agent. In FIG. 3, all stereocenters shown as L-amino acids; D-amino acids are also possible at all positions. R1 may comprise H, CH3. R2 may comprise H (alanine), OH (serine). R3 may comprise H, CH3, or a linear alkane with 2-9 carbons, mono- or polyunsaturated linear hydocarbons with 2-9 carbons, branched saturated or unsaturated hydrocarbons with length 2-9 carbons, or mono- or poly-unsaturated linear hydocarbons with terminal aromatic groups. R4 may comprise phenyl (phenylalanine), indole (tryptophan), or other aromatic group. R5 may comprise leucine, isoleucine, methionine, or phenylalanine. R6 may comprise H (alanine), OH (serine). X comprises a peptide sequence from 0-4 amino acids (for example PEHQ, PTHQ, PEFQ). Y comprises an imaging modality (e.g. fluorescent group (fluorescein, TAMRA, coumarin, etc), a radioactive group (group incorporating 18F, 14C, 3H), or a chelator for imaging metal (lanthanide, Tc, etc).

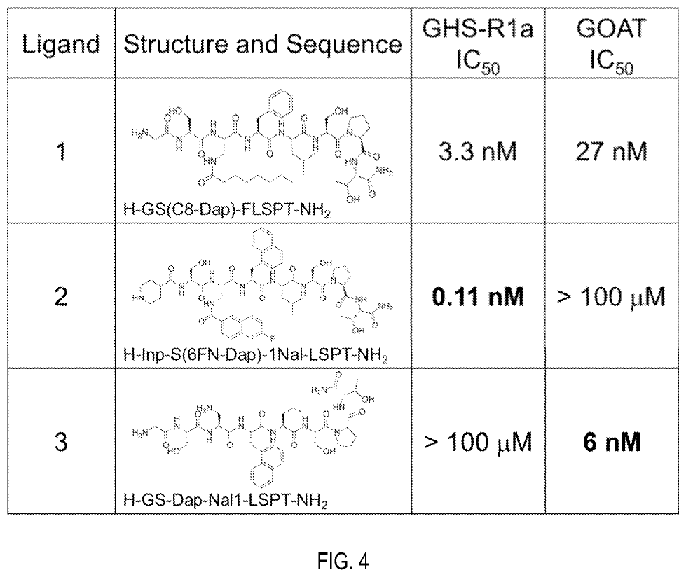

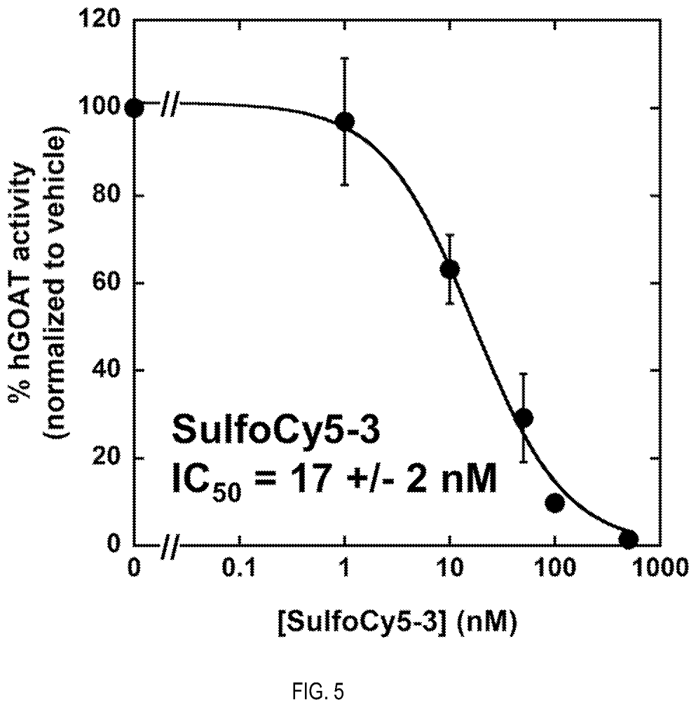

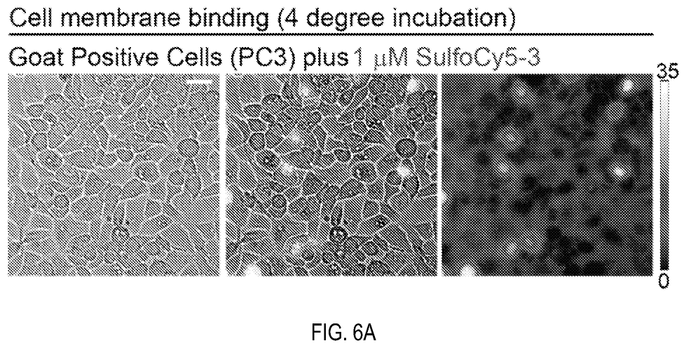

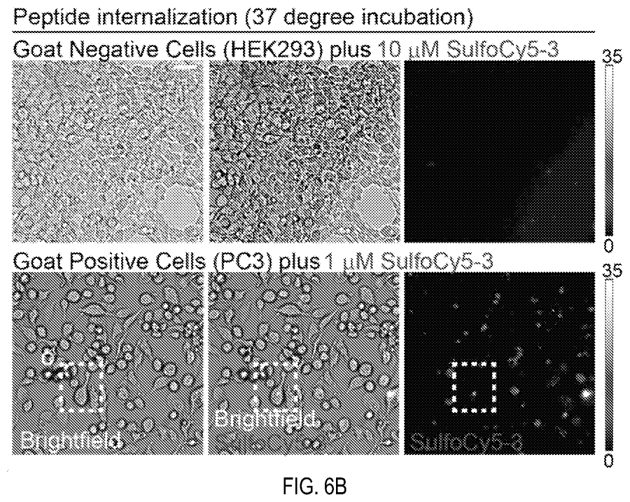

Referring to FIG. 4, the IC₅₀ values were determined for certain compounds of the present invention using three different ligands using GHS-R1A and GOAT assays. Referring to FIG. 5, SulfoCy5-3 inhibition studies demonstrated that the compound could be used to determine hGOAT binding. SulfoCy5-3 labeling of GOAT in PC3 prostate cancer cells and HEK293 cells was also used to determine cell membrane binding by incubating with the SulfoCy5-3 ligand and then imaging by fluorescence microscopy at 20× magnification (scale bar is 10 mM). In FIG. 6A, the incubation of PC3 cells at 4° C. to minimize membrane recycling led to plasma membrane binding. In FIG. 6B, the incubation of PC3 cells and HEK293 cells at 37° C. showed no labeling with the HEK293 cells and peptide label internalization with PC3 cells.

What is claimed is:

1. A method of imaging ghrelin O-acyltransferase (GOAT) activity, comprising the step of providing an imaging agent comprised of a peptide sequence including a ghrelin O-acyltransferase binding ligand and an imaging modality coupled to the peptide sequence, wherein the peptide sequence has a reduced binding affinity to a ghrelin receptor compared to ghrelin.

2. The method of claim 1, wherein the imaging agent has the formula 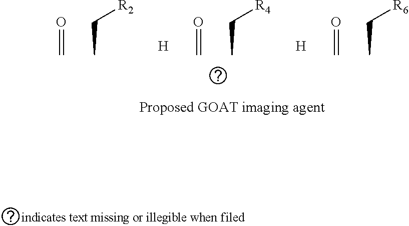
where R1 is selected from the group consisting of H and CH₃, R2 is selected from the group of consisting of H and OH, R3 is selected from the group consisting of H, CH₃, a linear alkane having two to nine carbons, a branched saturated hydrocarbon having two to nine carbons, an unsaturated hydrocarbon having two to nine carbons, a monounsaturated linear hydrocarbon having a terminal aromatic group, and a polyunsaturated linear hydrocarbon having a terminal aromatic group, R4 is selected from group consisting of a phenyl, an indole, and an aromatic group, R5 is selected from the group consisting of leucine, isoleucine, methionine, and phenylalanine, R6 is selected from the group consisting of H and OH, X is a peptide sequence, and Y is an imaging modality.

3. The method of claim 1, wherein the peptide sequence has the formula

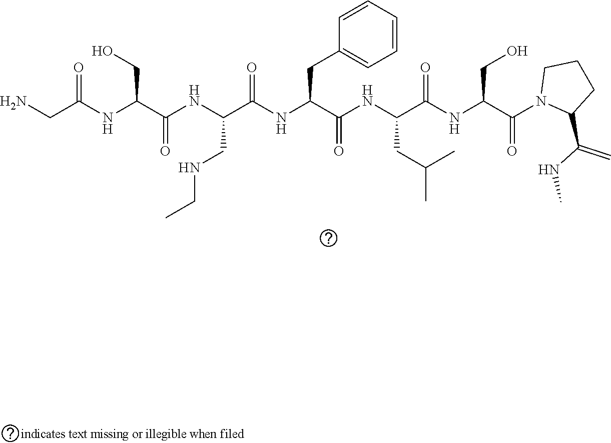

4. The method of claim 3, wherein the imaging modality is a fluorescent label.

5. The imaging agent of claim 3, wherein the imaging modality is a radioactive label.

6. The imaging agent of claim 3, wherein the imaging modality is a chelator.

7. A ghrelin O-acyltransferase (GOAT) imaging agent, comprising a peptide sequence including a ghrelin O-acyltransferase binding ligand and an imaging modality coupled to the peptide sequence, wherein the peptide sequence has a reduced binding affinity to a ghrelin receptor compared to ghrelin.

8. The imaging agent of claim 7, wherein the imaging agent has the formula 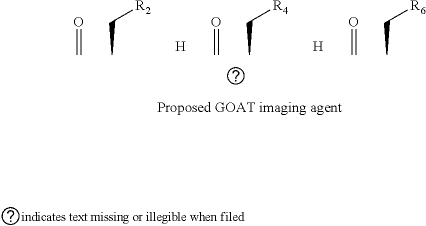
where R1 is selected from the group consisting of H and CH₃, R2 is selected from the group of consisting of H and OH, R3 is selected from the group consisting of H, CH₃, a linear alkane having two to nine carbons, a branched saturated hydrocarbon having two to nine carbons, an unsaturated hydrocarbon having two to nine carbons, a monounsaturated linear hydrocarbon having a terminal aromatic group, and a polyunsaturated linear hydrocarbon having a terminal aromatic group, R4 is selected from group consisting of a phenyl, an indole, and an aromatic group, R5 is selected from the group consisting of leucine, isoleucine, methionine, and phenylalanine, R6 is selected from the group consisting of H and OH, X is a peptide sequence, and Y is an imaging modality.

9. The imaging agent of claim 8, wherein the peptide sequence has the formula

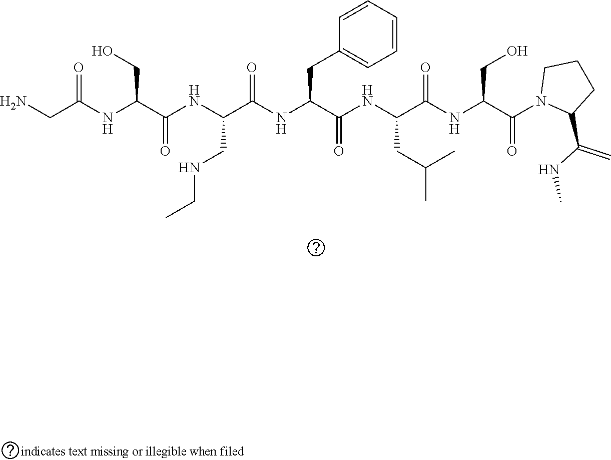

10. The imaging agent of claim 9, wherein the imaging modality is a fluorescent label.

11. The imaging agent of claim 9, wherein the imaging modality is a radioactive label.

12. The imaging agent of claim 9, wherein the imaging modality is a chelator.
